(19) United States
(12) Patent Application Publication
(10) Pub. No.: US 2025/0041373 A1
(43) Pub. Date: Feb. 6, 2025

(54) EXTRACTION METHOD OF HIBISCUS SABDARIFFA L. TAITUNG NO. 6, HIBISCUS SABDARIFFA L. TAITUNG NO. 6 EXTRACT USING THE SAME, AND USES THEREOF

(71) Applicant: WEL-BLOOM BIO-TECH CORPORATION, New Taipei City (TW)

(72) Inventors: CHIU-YUEH WANG, New Taipei City (TW); TING-TING LIU, New Taipei City (TW); YUN-HSIEN HSIEH, New Taipei City (TW); YUAN WEN, New Taipei City (TW)

(21) Appl. No.: 18/788,076

(22) Filed: Jul. 29, 2024

(30) Foreign Application Priority Data
Aug. 1, 2023 (TW) 112128724

(51) Int. Cl. A61K 36/185; A61K 8/9789; A61P 17/00; A61Q 19/00

(52) U.S. Cl. CPC A61K 36/5775; A61K 8/9789; A61P 17/00; A61Q 19/00; A61K 2236/15; A61K 2236/331; A61K 2800/82

(57) ABSTRACT

An extraction method of Hibiscus sabdariffa L. ‘TAITUNG NO. 6’, a Hibiscus sabdariffa L. ‘TAITUNG NO. 6’ extract using the same, and uses thereof are provided. The extraction method includes: providing the Hibiscus sabdariffa L. ‘TAITUNG NO. 6’ that is dried as a raw material for extraction; pulverizing the raw material; mixing the raw material that is pulverized with an extraction solvent for primary extraction, so as to obtain a Hibiscus sabdariffa L. ‘TAITUNG NO. 6’ primary extract; performing ultrasonic extraction on the Hibiscus sabdariffa L. ‘TAITUNG NO. 6’ primary extract, so as to obtain a Hibiscus sabdariffa L. ‘TAITUNG NO. 6’ extract; and filtering the Hibiscus sabdariffa L. ‘TAITUNG NO. 6’ extract.

### CROSS-REFERENCE TO RELATED PATENT APPLICATION

This application claims the benefit of priority to Taiwan Patent Application No. 112128724, filed on Aug. 1, 2023. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an extraction method, and more particularly to an extraction method of Hibiscus sabdariffa L. ‘TAITUNG NO. 6’, an extract using the same, and uses thereof.

### BACKGROUND OF THE DISCLOSURE

Hibiscus sabdariffa is an annual or biennial herb distributed in tropical and subtropical zones. The calyx of the Hibiscus sabdariffa is most commonly consumed by people, and is often made into preserves, jams, and tea.

It is known that the Hibiscus sabdariffa contains organic acids, carbohydrates, carotene, anthocyanins, etc. In addition, studies have shown that the Hibiscus sabdariffa has effects of anti-cancer, anti-inflammation, lowering cholesterol and triglycerides, regulating blood pressure, and protecting the liver.

Therefore, how to improve an extraction method, so as to enhance active components of the Hibiscus sabdariffa or its related varieties for increasing their efficacy or discover novel uses thereof, has become one of the important issues to be addressed in the related field.

### SUMMARY OF THE DISCLOSURE

In response to the above-referenced technical inadequacies, the present disclosure provides an extraction method of Hibiscus sabdariffa L. ‘TAITUNG NO. 6’, a Hibiscus sabdariffa L. ‘TAITUNG NO. 6’ extract using the same, and uses thereof.

In order to solve the above-mentioned problems, one of the technical aspects adopted by the present disclosure is to provide an extraction method of Hibiscus sabdariffa L. ‘TAITUNG NO. 6’. The extraction method includes: providing the Hibiscus sabdariffa L. ‘TAITUNG NO. 6’ that is dried as a raw material for extraction; pulverizing the raw material; mixing the raw material that is pulverized with an extraction solvent for primary extraction, so as to obtain a Hibiscus sabdariffa L. ‘TAITUNG NO. 6’ primary extract; performing ultrasonic extraction on the Hibiscus sabdariffa L. ‘TAITUNG NO. 6’ primary extract, so as to obtain a Hibiscus sabdariffa L. ‘TAITUNG NO. 6’ extract; and filtering the Hibiscus sabdariffa L. ‘TAITUNG NO. 6’ extract.

In order to solve the above-mentioned problems, another one of the technical aspects adopted by the present disclosure is to provide a Hibiscus sabdariffa L. ‘TAITUNG NO. 6’ extract prepared by the extraction method as described above.

In order to solve the above-mentioned problems, yet another one of the technical aspects adopted by the present disclosure is to provide a food composition that includes the Hibiscus sabdariffa L. ‘TAITUNG NO. 6’ extract as described above.

In order to solve the above-mentioned problems, still another one of the technical aspects adopted by the present disclosure is to provide a pharmaceutical composition that includes the Hibiscus sabdariffa L. ‘TAITUNG NO. 6’ extract as described above.

In order to solve the above-mentioned problems, still yet another one of the technical aspects adopted by the present disclosure is to provide a skin care cosmetic product that includes the Hibiscus sabdariffa L. ‘TAITUNG NO. 6’ extract as described above.

In order to solve the above-mentioned problems, still yet another one of the technical aspects adopted by the present disclosure is to provide a use of a Hibiscus sabdariffa L. ‘TAITUNG NO. 6’ extract for preparation of a pharmaceutical composition for skin care, in which the Hibiscus sabdariffa L. ‘TAITUNG NO. 6’ extract is prepared by the extraction method as described above.

Therefore, in the extraction method of the Hibiscus sabdariffa L. ‘TAITUNG NO. 6’, the Hibiscus sabdariffa L. ‘TAITUNG NO. 6’ extract using the same, and the uses thereof provided by the present disclosure, by virtue of “providing the Hibiscus sabdariffa L. ‘TAITUNG NO. 6’ that is dried as a raw material for extraction; pulverizing the raw material; mixing the raw material that is pulverized with an extraction solvent for primary extraction, so as to obtain a Hibiscus sabdariffa L. ‘TAITUNG NO. 6’ primary extract; performing ultrasonic extraction on the Hibiscus sabdariffa L. ‘TAITUNG NO. 6’ primary extract, so as to obtain a Hibiscus sabdariffa L. ‘TAITUNG NO. 6’ extract; and filtering the Hibiscus sabdariffa L. ‘TAITUNG NO. 6’ extract,” active components of the Hibiscus sabdariffa L. ‘TAITUNG NO. 6’ extract can be enhanced.

Further, the Hibiscus sabdariffa L. ‘TAITUNG NO. 6’ extract prepared by the above-mentioned technical solution has beneficial effects of inhibiting tyrosinase activity, decreasing a melanin content, increasing an elastin content, protecting cells from UVA damage, etc. That is, the Hibiscus sabdariffa L. ‘TAITUNG NO. 6’ extract prepared by the extraction method of the present disclosure has positive effects on skin care.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a flowchart of an extraction method of Hibiscus sabdariffa L. ‘TAITUNG NO. 6’ according to one embodiment of the present disclosure;

FIG. 2 is a flowchart of the extraction method of the Hibiscus sabdariffa L. ‘TAITUNG NO. 6’ according to another embodiment of the present disclosure;

FIG. 3 is an HPLC (high-performance liquid chromatography) profile of a Hibiscus sabdariffa L. ‘TAITUNG NO. 6’ extract prepared by the extraction method of the Hibiscus sabdariffa L. ‘TAITUNG NO. 6’ according to a first embodiment of the present disclosure;

FIG. 4 is an HPLC profile of an extract of Hibiscus sabdariffa L. ‘TAITUNG NO. 3’ (a commercial variety of Hibiscus sabdariffa) according to the first embodiment of the present disclosure;

FIG. 5 is an analysis diagram illustrating an effect of the Hibiscus sabdariffa L. ‘TAITUNG NO. 6’ extract on tyrosinase activity of melanoma cells (in which no repetition of the English letters on bar charts of each group indicates a significant difference therebetween (P≤0.05)) according to a second embodiment of the present disclosure;

FIG. 6 is an analysis diagram illustrating an effect of the Hibiscus sabdariffa L. ‘TAITUNG NO. 6’ extract on a melanin content of the melanoma cells (in which no repetition of the English letters on bar charts of each group indicates a significant difference therebetween (P≤0.05)) according to the second embodiment of the present disclosure;

FIG. 7 is an analysis diagram illustrating an effect of the Hibiscus sabdariffa L. ‘TAITUNG NO. 6’ extract on increasing an elastin content of fibroblasts (in which no repetition of the English letters on bar charts of each group indicates a significant difference therebetween (P≤0.05)) according to a third embodiment of the present disclosure; and

FIG. 8 is an analysis diagram illustrating an effect of the Hibiscus sabdariffa L. ‘TAITUNG NO. 6’ extract on improving an anti-UV ability of the fibroblasts (in which no repetition of the English letters on bar charts of each group indicates a significant difference therebetween (P≤0.05)) according to a fourth embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

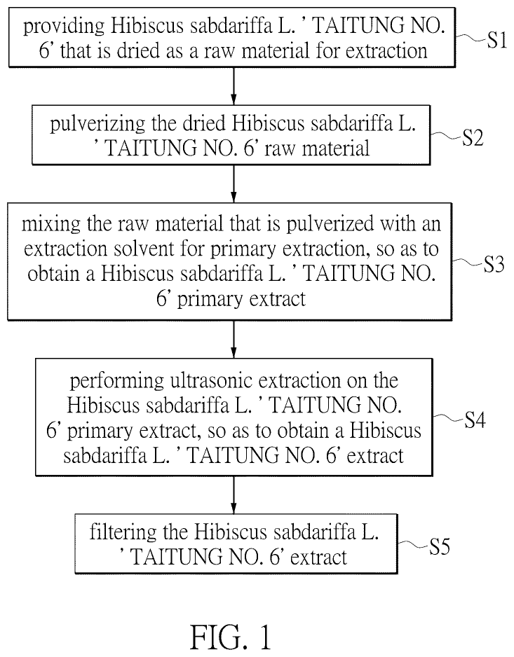

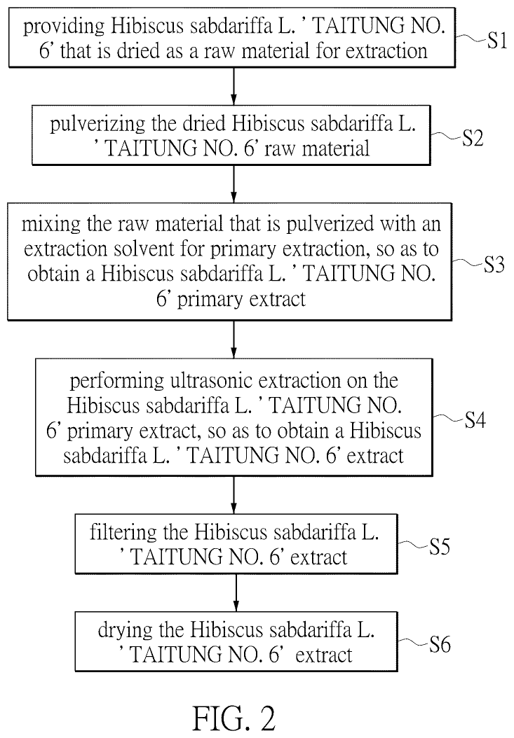

Referring to FIG. 1 and FIG. 2, the present disclosure provides an extraction method of Hibiscus sabdariffa L. ‘TAITUNG NO. 6’, which at least includes: providing Hibiscus sabdariffa L. ‘TAITUNG NO. 6’ that is dried as a raw material for extraction (step S1); pulverizing the raw material (step S2); mixing the raw material that is pulverized with an extraction solvent for primary extraction, so as to obtain a Hibiscus sabdariffa L. ‘TAITUNG NO. 6’ primary extract (step S3); performing ultrasonic extraction on the Hibiscus sabdariffa L. ‘TAITUNG NO. 6’ primary extract, so as to obtain a Hibiscus sabdariffa L. ‘TAITUNG NO. 6’ extract (step S4); and filtering the Hibiscus sabdariffa L. ‘TAITUNG NO. 6’ extract (step S5).

In one particular embodiment, the extraction method of the present disclosure further includes: drying the Hibiscus sabdariffa L. ‘TAITUNG NO. 6’ extract (step S6).

In one particular embodiment, the Hibiscus sabdariffa L. ‘TAITUNG NO. 6’ extract of the present disclosure can be ingested in the form of food. The Hibiscus sabdariffa L. ‘TAITUNG NO. 6’ extract can be prepared into a food composition of the Hibiscus sabdariffa L. ‘TAITUNG NO. 6’ extract in liquid, solid, granular, powdered, jelly, or gelatinous form. The food composition of the Hibiscus sabdariffa L. ‘TAITUNG NO. 6’ extract can be combined with an excipient or a flavoring agent to improve flavor or convenience of consumption.

The excipient that can be combined with the food composition of the Hibiscus sabdariffa L. ‘TAITUNG NO. 6’ extract includes, for example, corn starch, potato starch, dextrin, lactose, glucose, fructose, sorbitol, xylitol, and mannitol, and can be adjusted according to requirements and practical applications, but the present disclosure is not limited thereto.

The flavoring agent that can be combined with the food composition of the Hibiscus sabdariffa L. ‘TAITUNG NO. 6’ extract includes, for example, a fruit juice extract, sucralose, mannose, xylitol, citric acid, malic acid, and tartaric acid, and can be adjusted according to requirements and practical applications, but the present disclosure is not limited thereto.

In one particular embodiment, the Hibiscus sabdariffa L. ‘TAITUNG NO. 6’ extract of the present disclosure has beneficial effects of inhibiting tyrosinase activity, decreasing a melanin content, increasing an elastin content, protecting cells from UVA damage, etc. Accordingly, the Hibiscus sabdariffa L. ‘TAITUNG NO. 6’ extract of the present disclosure can be further formulated into a pharmaceutical composition for skin care. The present disclosure further provides a pharmaceutical composition that includes a therapeutically effective amount of the Hibiscus sabdariffa L. ‘TAITUNG NO. 6’ extract and one or more pharmaceutically acceptable carriers.

For the purpose of delivery and absorption, the therapeutically effective amount of the Hibiscus sabdariffa L. ‘TAITUNG NO. 6’ extract of the present disclosure can be formulated with the pharmaceutically acceptable carrier into a suitable form of the pharmaceutical composition for skin care. Depending on a route of administration, the pharmaceutical composition of the Hibiscus sabdariffa L. ‘TAITUNG NO. 6’ extract of the present disclosure preferably includes 0.1% to 100% by weight of the Hibiscus sabdariffa L. ‘TAITUNG NO. 6’ extract.

In one particular embodiment, the pharmaceutically acceptable carrier can be an acceptable carrier, diluent, or excipient that is compatible with other components in the formulation and is not harmful to an individual to whom the pharmaceutical composition is to be administrated. In the present disclosure, any carrier, diluent, or excipient commonly known or used in the related field can be used depending on requirements for the pharmaceutical formulation. According to the present disclosure, the pharmaceutical composition can be administrated by any appropriate route, which includes, but is not limited to, an oral, a rectal, a nasal, a topical, a vaginal, or a parenteral route. In a specific embodiment of the present disclosure, the pharmaceutical composition is formulated for oral administration. Such preparations can be prepared by any known method in the field of pharmacy.

In one particular embodiment, the pharmaceutical composition of the Hibiscus sabdariffa L. ‘TAITUNG NO. 6’ extract of the present disclosure can be in the form of tablets, pills, powders, oral tablets, sachets, lozenges, elixirs, suspensions, lotions, solutions, syrups, soft and hard gelatin capsules, suppositories, sterile injections, and packaged powders, but the present disclosure is not limited thereto.

In one particular embodiment, the Hibiscus sabdariffa L. ‘TAITUNG NO. 6’ extract of the present disclosure can be further formulated for purposes of manufacturing skin care cosmetic products, which have whitening, anti-allergic, and anti-aging effects.

In one particular embodiment, when being formulated for purposes of manufacturing the skin care cosmetic products, the Hibiscus sabdariffa L. ‘TAITUNG NO. 6’ extract of the present disclosure can be further formulated into an external preparation suitable for topical application to the skin, such as, but not limited to, emulsions, gels, creams, ointments, patches, liniments, powders, aerosols, sprays, lotions, magmas, pastes, foams, drops, suspensions, and bandages.

### First Embodiment

Referring to FIG. 1 and FIG. 2, a first embodiment of the present disclosure provides an extraction method of Hibiscus sabdariffa L. ‘TAITUNG NO. 6’, which includes the following steps.
Step S1: providing dried Hibiscus sabdariffa L. ‘TAITUNG NO. 6’ as a raw material for extraction. In one particular embodiment, the raw material for extraction is dried Hibiscus sabdariffa L. ‘TAITUNG NO. 6’. Preferably, the raw material for extraction is the dried Hibiscus sabdariffa L. ‘TAITUNG NO. 6’ having a moisture content of less than 10%. Drying of the Hibiscus sabdariffa L. ‘TAITUNG NO. 6’ can be carried out by sun drying, hot air drying, cold air drying, freeze drying, vacuum drying, etc., and can be adjusted according to requirements and practical applications. However, the present disclosure is not limited thereto. It should be noted that prior to drying, the Hibiscus sabdariffa L. ‘TAITUNG NO. 6’ can be subjected to a pre-treatment, so as to enhance the drying effect or prevent deterioration of ingredients in the Hibiscus sabdariffa L. ‘TAITUNG NO. 6’ during the drying process. For example, blanching can be performed by scalding the raw material for extraction in boiling water, thereby inhibiting enzyme activities contained in the Hibiscus sabdariffa L. ‘TAITUNG NO. 6’. The pre-treatment can be adjusted according to requirements and practical applications, but the present disclosure is not limited thereto.
Step S2: pulverizing the raw material. In one particular embodiment, the raw material for extraction can be blended into fine powder by a grinder and homogenized through a 100-mesh sieve, so as to obtain a Hibiscus sabdariffa L. ‘TAITUNG NO. 6’ powder. However, the present disclosure is not limited thereto. A homogenizer or other homogenization methods can be selected according to conventional techniques, such that the raw material for extraction is pulverized. In addition, a mesh number of the sieve can also be selected according to practical applications, but the present disclosure is not limited thereto.
Step S3: mixing the Hibiscus sabdariffa L. ‘TAITUNG NO. 6’ powder with an extraction solvent for primary extraction. In one particular embodiment, the extraction solvent can be water and/or a water-soluble organic solvent. The water-soluble organic solvent can be one of methanol, ethanol, n-propanol, isopropanol, butanol, ethylene glycol, propylene glycol, and glycerol, or any mixture thereof, but the present disclosure is not limited thereto. In one exemplary embodiment, the extraction solvent of the Hibiscus sabdariffa L. ‘TAITUNG NO. 6’ is water, and a mixing ratio of the Hibiscus sabdariffa L. ‘TAITUNG NO. 6’ powder to water ranges from 1:1 to 1:30 (preferably ranges from 1:10 to 1:20, and is more preferably 1:15), but the present disclosure is not limited thereto. Further, after the Hibiscus sabdariffa L. ‘TAITUNG NO. 6’ powder and the extraction solvent are mixed, the Hibiscus sabdariffa L. ‘TAITUNG NO. 6’ powder can be uniformly dispersed in the extraction solvent by standing, stirring, shaking, etc. The mixing method can be adjusted according to requirements and practical applications, but the present disclosure is not limited thereto. In addition, a mixture of the Hibiscus sabdariffa L. ‘TAITUNG NO. 6’ powder and the extraction solvent can be heated to obtain a Hibiscus sabdariffa L. ‘TAITUNG NO. 6’ primary extract. For example, the primary extraction can be conducted at 100° C., 95° C., 90° C., 85° C., 80° C., 75° C., 70° C., 65° C., 60° C., 55° C., 50° C., 45° C., 40° C., 35° C., etc. In one exemplary embodiment, the primary extraction is conducted at 60° C. In addition, a duration of the primary extraction can be 15 minutes, 30 minutes, 45 minutes, 60 minutes, 75 minutes, 90 minutes, 120 minutes, 180 minutes, 240 minutes, etc. In one exemplary embodiment, the duration of the primary extraction ranges from 15 minutes to 60 minutes, and is preferably 30 minutes. It should be noted that, after the above-mentioned primary extraction step, the Hibiscus sabdariffa L. ‘TAITUNG NO. 6’ primary extract can be directly used as a Hibiscus sabdariffa L. ‘TAITUNG NO. 6’ extract, but the present disclosure is not limited thereto.
Step S4: performing ultrasonic extraction on the Hibiscus sabdariffa L. ‘TAITUNG NO. 6’ primary extract. Specifically, after step S3, the Hibiscus sabdariffa L. ‘TAITUNG NO. 6’ primary extract can be pressed and filtered, and the remaining solid residue that cannot be filtered (hereinafter referred to as a “Hibiscus sabdariffa L. ‘TAITUNG NO. 6’ residue”) is subjected to the ultrasonic extraction. In one particular embodiment, the extraction solvent can be water and/or a water-soluble organic solvent. The water-soluble organic solvent can be one of methanol, ethanol, n-propanol, isopropanol, butanol, ethylene glycol, propylene glycol, and glycerol, or any mixture thereof, but the present disclosure is not limited thereto. In one exemplary embodiment, the extraction solvent of the Hibiscus sabdariffa L. ‘TAITUNG NO. 6’ residue is water, and a mixing ratio of the Hibiscus sabdariffa L. ‘TAITUNG NO. 6’ residue to water ranges from 1:1 to 1:30, preferably ranges from 1:10 to 1:20, and more preferably ranges from 1:2.5 to 1:5, but the present disclosure is not limited thereto. In addition, a mixture of the Hibiscus sabdariffa L. ‘TAITUNG NO. 6’ residue and the extraction solvent can be heated and subjected to the ultrasonic extraction, so as to obtain a Hibiscus sabdariffa L. ‘TAITUNG NO. 6’ ultrasonic extract. For example, the ultrasonic extraction can be conducted at 100° C., 95° C., 90° C., 85° C., 80° C., 75° C., 70° C., 65° C., 60° C., 55° C., 50° C., 45° C., 40° C., 35° C., etc. In one exemplary embodiment, the ultrasonic extraction can be conducted at 60° C. In one particular embodiment, the mixture of the Hibiscus sabdariffa L. ‘TAITUNG NO. 6’ residue and the extraction solvent can be subjected to the ultrasonic extraction with a power of 200 W and a frequency of 40 KHz. In addition, an ultrasonic extraction time can be adjusted according to requirements and practical applications, but the present disclosure is not limited thereto. For example, the ultrasonic extraction time can be 15 minutes, 30 minutes, 45 minutes, 60 minutes, 75 minutes, 90 minutes, 120 minutes, 180 minutes, 240 minutes, etc. In one exemplary embodiment, the ultrasonic extraction time ranges from 15 minutes to 60 minutes, and is preferably 30 minutes. It should be noted that, after the above-mentioned ultrasonic extraction step, the Hibiscus sabdariffa L. ‘TAITUNG NO. 6’ ultrasonic extract can be directly used as the Hibiscus sabdariffa L. ‘TAITUNG NO. 6’ extract, but the present disclosure is not limited thereto.
Step S5: filtering the Hibiscus sabdariffa L. ‘TAITUNG NO. 6’ extract. As described above, the Hibiscus sabdariffa L. ‘TAITUNG NO. 6’ primary extract obtained after step S3 can be directly used as the Hibiscus sabdariffa L. ‘TAITUNG NO. 6’ extract, or can be subjected to a filtration process described in step S5. In addition, if step S3 is followed by step S4, the Hibiscus sabdariffa L. ‘TAITUNG NO. 6’ ultrasonic extract can be mixed with the Hibiscus sabdariffa L. ‘TAITUNG NO. 6’ primary extract before proceeding to the filtration process described in step S5. Alternatively, the Hibiscus sabdariffa L. ‘TAITUNG NO. 6’ ultrasonic extract and the Hibiscus sabdariffa L. ‘TAITUNG NO. 6’ primary extract can be separately subjected to the filtration process described in step S5 before being mixed together. However, the present disclosure is not limited thereto. In one particular embodiment, the filtration process can be conducted by pressing and filtering the Hibiscus sabdariffa L. ‘TAITUNG NO. 6’ primary extract and/or the Hibiscus sabdariffa L. ‘TAITUNG NO. 6’ ultrasonic extract, so as to obtain the Hibiscus sabdariffa L. ‘TAITUNG NO. 6’ extract. For example, press filtration can be implemented by using a filter press, but the present disclosure is not limited thereto. Further, the filtration process can be conducted by using a filter cloth or a filter mesh. In one exemplary embodiment, the filter cloth can be a 20 mesh, 40 mesh, 60 mesh, 80 mesh, 100 mesh, 200 mesh, 300 mesh, or 400 mesh filter cloth. Preferably, the 100 mesh filter cloth is adopted in the embodiments of the present disclosure, but the present disclosure is not limited thereto. It should be noted that, in order to obtain the Hibiscus sabdariffa L. ‘TAITUNG NO. 6’ extract, centrifugation can also be adopted to separate out the remaining solid residue, but the present disclosure is not limited thereto.

It should be noted that the Hibiscus sabdariffa L. ‘TAITUNG NO. 6’ extract can be further subjected to membrane filtration, heat treatment, protease inhibitor treatment, or preservative treatment, so as to maintain integrity and activity of components of the Hibiscus sabdariffa L. ‘TAITUNG NO. 6’ extract. However, the present disclosure is not limited thereto.
Step S6: drying the Hibiscus sabdariffa L. ‘TAITUNG NO. 6’ extract. It should be noted that the Hibiscus sabdariffa L. ‘TAITUNG NO. 6’ extract obtained after step S5 can also be directly used without proceeding to step S6. In one particular embodiment, the Hibiscus sabdariffa L. ‘TAITUNG NO. 6’ extract can be further dried, such that the Hibiscus sabdariffa L. ‘TAITUNG NO. 6’ extract is in a powdery form. The drying method can be, for example, but not limited to, spray drying, vacuum freeze drying, microwave vacuum drying, and low-temperature cold-air drying.

It should be noted that, as long as the extraction efficiency and efficacy of the Hibiscus sabdariffa L. ‘TAITUNG NO. 6’ extract can be satisfied, the Hibiscus sabdariffa L. ‘TAITUNG NO. 6’ extract of the present disclosure can also be extracted by conventional extraction methods. For example, the extraction method can be conducted by adjusting the pH of the extraction environment and/or by adding extraction aids, but the present disclosure is not limited thereto.

In one particular embodiment, the Hibiscus sabdariffa L. ‘TAITUNG NO. 6’ extract is further subjected to a component analysis. A high-performance liquid chromatography (HPLC) instrument used in the present embodiment is the Agilent 1100 series system, which includes Degasser G1379A, QuatPump G1311A, an Agilent manual FL-injection valve injector, and a VWD G1314A variable wavelength detector. The analysis conditions are shown in Table 1 below.

TABLE 1
(conditions for performing the HPLC analysis).

| Column | Mightysil RP-18 GP 250-4.5 (5 μm) | | | |
|---|---|---|---|---|
| Column | 4.6 mm I.D. × 250 mm | | | |
| dimension | | | | |
| Temperature | Room temperature | | | |
| Detection | 522 nm | | | |
| wavelength | | | | |
| | | | | trifluoroacetic |
| | Time | methanol | water | acid |
| | (min) | (%) | (%) | (%) |
| Mobile phase | 0.0→20.0 | 50→50 | 49.99→49.99 | 0.01→0.01 |
| Flow velocity | 1 | | | |
| (mL/min) | | | | |
| Injection | 20 μL | | | |
| volume | | | | |

In addition, in the present embodiment, cyanidin and delphinidin are used as standards, and are diluted to 0.025 mg/L, 0.05 mg/L, 0.1 mg/L, 0.2 mg/L, and 0.4 mg/L as standard solutions for the HPLC analysis. The analysis results are used to create a standard curve, which can be applied to estimate contents of cyanidin and delphinidin in the Hibiscus sabdariffa L. ‘TAITUNG NO. 6’ extract.

In the present embodiment, an analysis is conducted to compare an extract of a conventional commercial variety, Hibiscus sabdariffa L. ‘TAITUNG NO. 3’ (hereinafter referred to as “the control group”), and the Hibiscus sabdariffa L. ‘TAITUNG NO. 6’ extract obtained by the extraction method of the Hibiscus sabdariffa L. ‘TAITUNG NO. 6’ of the present disclosure (hereinafter referred to as “the experimental group”). An extraction method and an HPLC analysis of the control group are the same as those of the experimental group, which will not be reiterated herein.

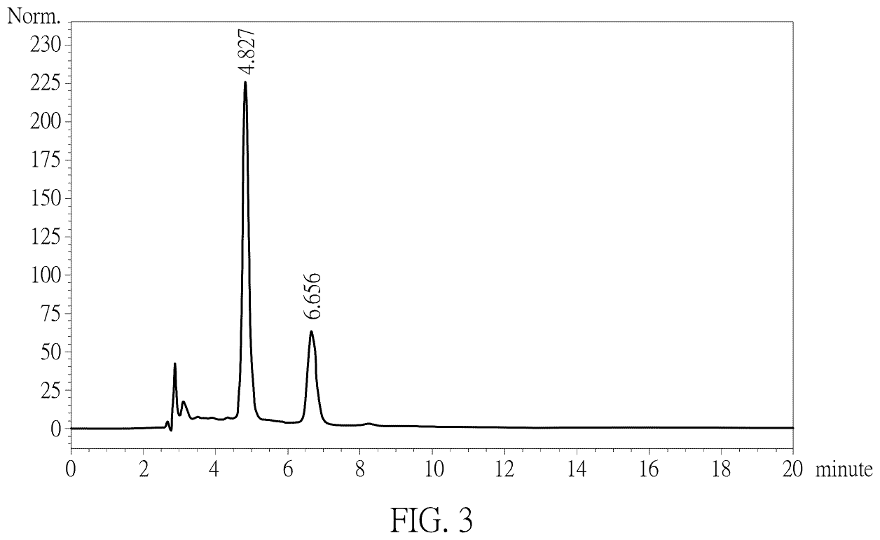

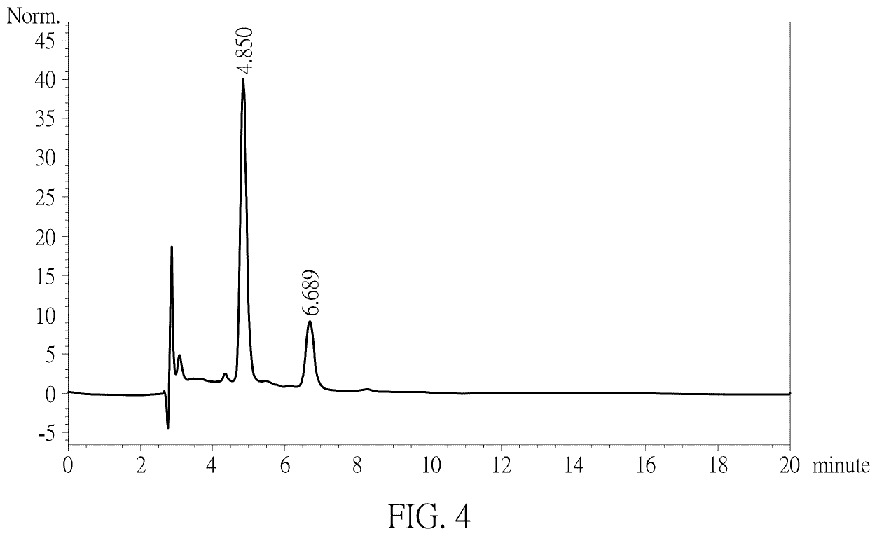

As shown in FIG. 3 and FIG. 4, the extract of the control group and the extract of the experimental group are subjected to the HPLC analysis, and HPLC profiles of the components are substantially the same. In addition, a calculation using the standard curve shows that the extract of the control group contains 0.26 μg/mL of cyanidin and 0.9 μg/mL of delphinidin, and the extract of the experimental group contains 2 μg/mL of cyanidin and 5 μg/mL of delphinidin. These results indicate that cyanidin and delphinidin contained in the Hibiscus sabdariffa L. ‘TAITUNG NO. 6’ extract of the present disclosure are much more than those contained in the extract of the conventional commercial variety of Hibiscus sabdariffa L.

However, the aforementioned details are disclosed for exemplary purposes only, and are not meant to limit the scope of the present disclosure.

### Second Embodiment

In the present embodiment, an ability of the Hibiscus sabdariffa L. ‘TAITUNG NO. 6’ extract to inhibit the tyrosinase activity is evaluated by tyrosinase activity assay, and a method thereof is as follows.

It should be noted that a cell survival analysis has already been conducted to determine whether or not concentrations of the Hibiscus sabdariffa L. ‘TAITUNG NO. 6’ extract adopted in the present embodiment are toxic to melanoma cells, and the adopted concentrations (i.e., 200 μg/mL, 400 μg/mL, and 800 μg/mL) of the Hibiscus sabdariffa L. ‘TAITUNG NO. 6’ extract are determined to be not cytotoxic (not shown in the figures).

B16-F10 melanoma cells (1×10⁵) are seeded in a 24-well culture plate and cultured at 37° C. for 24 hours. After being treated with various concentrations of the Hibiscus sabdariffa L. ‘TAITUNG NO. 6’ extract (the Hibiscus sabdariffa L. ‘TAITUNG NO. 6’ extract described below is the Hibiscus sabdariffa L. ‘TAITUNG NO. 6’ extract obtained after step S6 described above) for 48 hours, these cells are rinsed with phosphate-buffered saline (PBS) and collected by centrifugation at 10,000 g for 20 minutes at 4° C., so as to obtain a cell pellet. The cell pellet is resuspended in PBS containing 1% of Triton X-100 and 100 μM of PMSF (phenylmethylsulfonyl fluoride), and is then centrifuged at 17,500 g for 10 minutes at 4° C. to remove cell debris. 90 μl of cell lysate is added to 10 μl of L-Dopa (2 mg/ml), and is then incubated at 37° C. for 20 minutes to 30 minutes. Afterward, a spectrophotometer is used to measure absorbance at 490 nm. In addition, in the present embodiment, kojic acid is used as a positive control group, in which the cell treatment method and the tyrosinase activity assay are the same as those described above, and will not be reiterated herein.

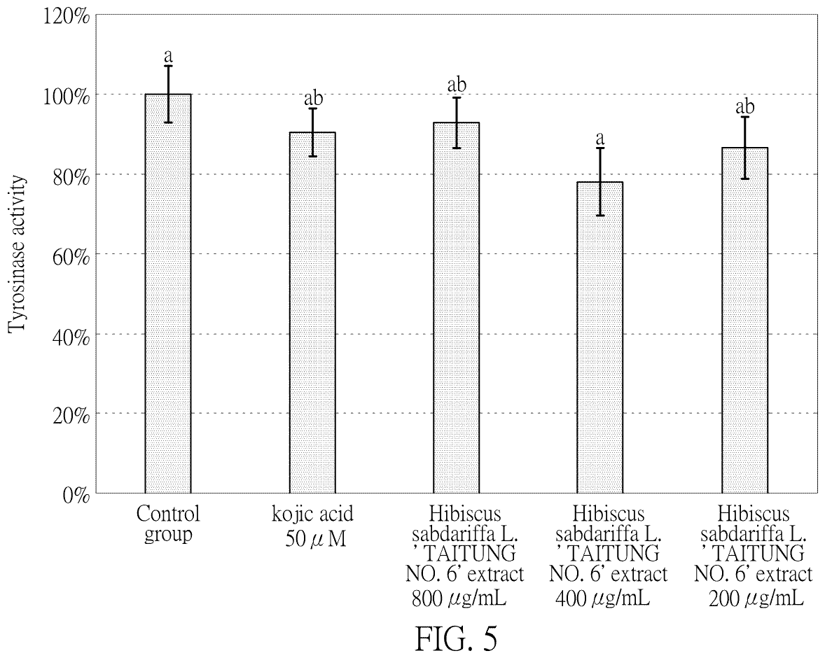

As shown in FIG. 5, when the melanoma cells are treated with the kojic acid, the tyrosinase activity of the melanoma cells is significantly inhibited. On the other hand, when the melanoma cells are treated with 200 μg/mL, 400 μg/mL, or 800 μg/mL of the Hibiscus sabdariffa L. ‘TAITUNG NO. 6’ extract, the tyrosinase activity of the melanoma cells can also be significantly inhibited. It should be noted that 400 μg/mL of the Hibiscus sabdariffa L. ‘TAITUNG NO. 6’ extract has a more significant inhibitory effect than the kojic acid (as shown in FIG. 5).

Further, in the present embodiment, an ability of the Hibiscus sabdariffa L. ‘TAITUNG NO. 6’ extract to inhibit melanin production is evaluated by a melanin content analysis, and a method thereof is as follows.

B16-F10 melanoma cells (1×10⁵) are seeded in a 24-well culture plate and treated with 500 nM α-melanocyte stimulating hormone (α-MSH) at 37° C. for 24 hours. Then, these cells are treated with various concentrations of the Hibiscus sabdariffa L. ‘TAITUNG NO. 6’ extract for 48 hours, and are treated with trypsin after being rinsed with PBS. A cell pellet is collected by centrifugation at 10,000 g for 20 minutes at a room temperature, and is then lysed with 1N NaOH for 60 minutes. The melanin content is analyzed by comparing an absorbance value of a test sample at 405 nm with a standard curve obtained by using a synthetic melanin.

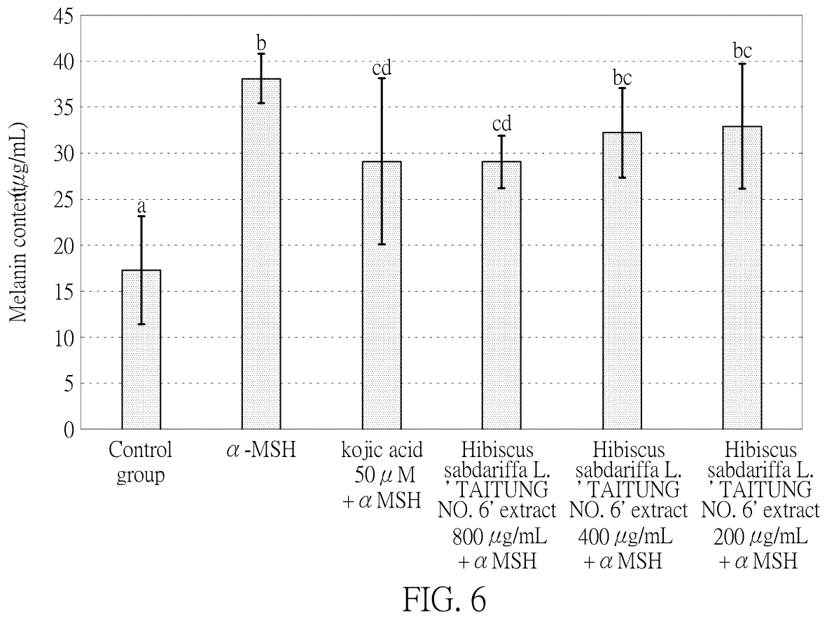

As shown in FIG. 6, when the melanoma cells are treated with α-MSH, the melanin content of the melanoma cells is significantly increased by about two times as compared to that of the untreated control group, and is significantly decreased by using the kojic acid for co-treatment. On the other hand, when the melanoma cells are treated with 800 μg/mL of the Hibiscus sabdariffa L. ‘TAITUNG NO. 6’ extract, the melanin content of the melanoma cells can also be significantly decreased.

However, the aforementioned details are disclosed for exemplary purposes only, and are not meant to limit the scope of the present disclosure.

### Third Embodiment

In the present embodiment, the potential of the Hibiscus sabdariffa L. ‘TAITUNG NO. 6’ extract on enhancing the elastin content is evaluated by an elastin content analysis, and a method thereof is as follows.

It should be noted that the cell survival analysis has already been conducted to determine whether or not concentrations of the Hibiscus sabdariffa L. ‘TAITUNG NO. 6’ extract adopted in the present embodiment are toxic to fibroblasts, and the adopted concentrations (i.e., 100 μg/mL, 200 μg/mL, 400 μg/mL, and 800 μg/mL) of the Hibiscus sabdariffa L. ‘TAITUNG NO. 6’ extract are determined to be not cytotoxic (not shown in the figures).

CCD966SK fibroblasts (4×10⁵) are seeded in a 24-well culture plate and cultured at 37° C. for 24 hours. Then, these cells are treated with various concentrations of the Hibiscus sabdariffa L. ‘TAITUNG NO. 6’ extract for 48 hours, and are treated with trypsin after being rinsed with PBS. A cell pellet collected by centrifugation at 2,000 g for 10 minutes at 4° C. is subjected to ultrasonication, and then a supernatant is collected after centrifugation at 10,000 g for 10 minutes at 4° C. Afterward, the elastin content is analyzed by using the Human Elastin ELISA kit (ab239433).

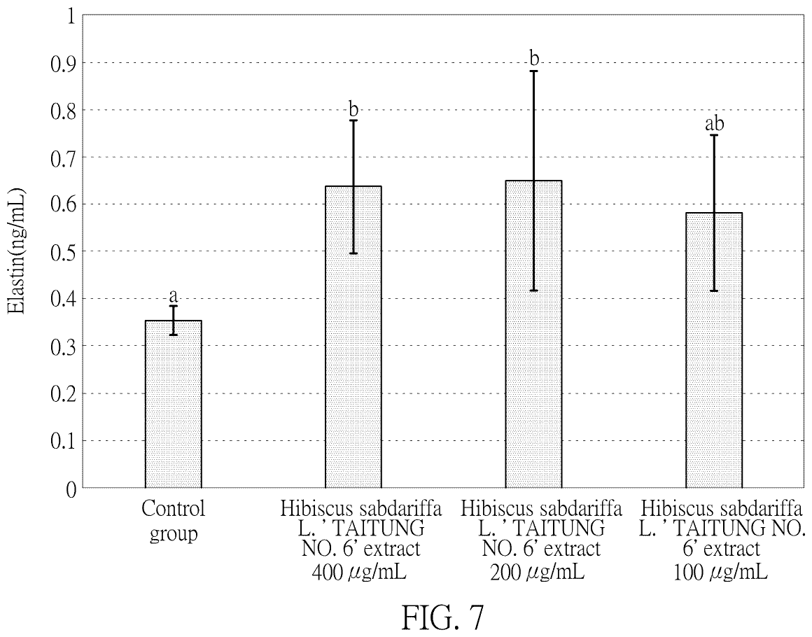

As shown in FIG. 7, when the fibroblasts are treated with 200 μg/mL or 400 μg/mL of the Hibiscus sabdariffa L. ‘TAITUNG NO. 6’ extract, the elastin content of the fibroblasts is significantly increased. These results indicate that the Hibiscus sabdariffa L. ‘TAITUNG NO. 6’ extract of the present disclosure has the potential to increase the elastin content.

However, the aforementioned details are disclosed for exemplary purposes only, and are not meant to limit the scope of the present disclosure.

### Fourth Embodiment

In the present embodiment, an anti-UV ability of the Hibiscus sabdariffa L. ‘TAITUNG NO. 6’ extract is evaluated by a UVA protection test, and a method thereof is as follows.

CCD966SK fibroblasts (8×10⁵) are seeded in a 96-well culture plate and cultured at 37° C. for 24 hours. Then, these cells are treated with various concentrations of the Hibiscus sabdariffa L. ‘TAITUNG NO. 6’ extract for 24 hours, and are incubated in 100 μl of MEM-α (phenol red-free) after being rinsed with PBS. Afterward, these cells are placed in VILBER-365 to be irradiated at 12 J/cm² for 60 minutes, and then are cultured for 24 hours. The cell viability is analyzed by using MTT assay.

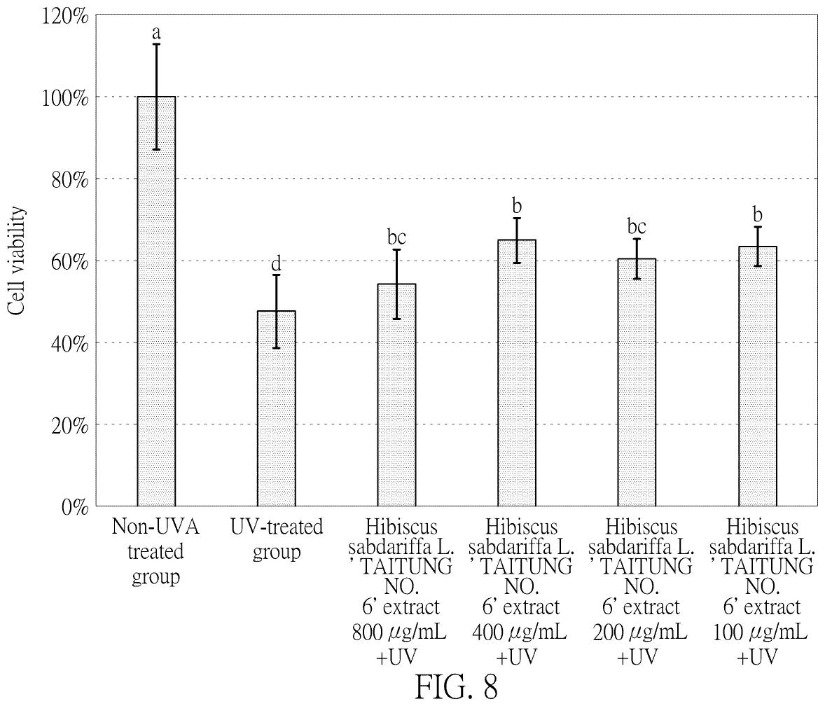

As shown in FIG. 8, when the fibroblasts are irradiated with UVA, the cell viability is significantly decreased to about 50% of the cell viability of a non-UVA treated control group. On the other hand, when the fibroblasts are treated with 100 μg/mL, 200 μg/mL, 400 μg/mL, or 800 μg/mL of the Hibiscus sabdariffa L. ‘TAITUNG NO. 6’ extract, the cell viability can be significantly increased.

However, the aforementioned details are disclosed for exemplary purposes only, and are not meant to limit the scope of the present disclosure.

### Beneficial Effects of the Embodiments

In conclusion, in the extraction method of the Hibiscus sabdariffa L. ‘TAITUNG NO. 6’, the Hibiscus sabdariffa L. ‘TAITUNG NO. 6’ extract using the same and the uses thereof provided by the present disclosure, by virtue of “providing the Hibiscus sabdariffa L. ‘TAITUNG NO. 6’ that is dried as the raw material for extraction; pulverizing the raw material; mixing the raw material that is pulverized with an extraction solvent for primary extraction, so as to obtain the Hibiscus sabdariffa L. ‘TAITUNG NO. 6’ primary extract; performing ultrasonic extraction on the Hibiscus sabdariffa L. ‘TAITUNG NO. 6’ primary extract, so as to obtain the Hibiscus sabdariffa L. ‘TAITUNG NO. 6’ extract; and filtering the Hibiscus sabdariffa L. ‘TAITUNG NO. 6’ extract,” active components of the Hibiscus sabdariffa L. ‘TAITUNG NO. 6’ extract can be enhanced.

Further, the Hibiscus sabdariffa L. ‘TAITUNG NO. 6’ extract prepared by the above-mentioned technical solution has beneficial effects of inhibiting the tyrosinase activity, decreasing the melanin content, increasing the elastin content, protecting cells from the UVA damage, etc. That is, the Hibiscus sabdariffa L. ‘TAITUNG NO. 6’ extract prepared by the extraction method of the present disclosure has positive effects on skin care.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An extraction method of Hibiscus sabdariffa L. ‘TAITUNG NO. 6’, comprising:
providing the Hibiscus sabdariffa L. ‘TAITUNG NO. 6’ that is dried as a raw material for extraction;
pulverizing the raw material;
mixing the raw material that is pulverized with an extraction solvent for primary extraction, so as to obtain a Hibiscus sabdariffa L. ‘TAITUNG NO. 6’ primary extract;
performing ultrasonic extraction on the Hibiscus sabdariffa L. ‘TAITUNG NO. 6’ primary extract, so as to obtain a Hibiscus sabdariffa L. ‘TAITUNG NO. 6’ extract; and
filtering the Hibiscus sabdariffa L. ‘TAITUNG NO. 6’ extract.

2. The extraction method according to claim 1, wherein a moisture content of the Hibiscus sabdariffa L. ‘TAITUNG NO. 6’ that is dried is less than 10%.

3. The extraction method according to claim 1, wherein the extraction solvent is water.

4. The extraction method according to claim 3, wherein a ratio of the raw material that is pulverized to the water is 1:15.

5. The extraction method according to claim 4, wherein the primary extraction is conducted at 60° C. for 15 minutes to 60 minutes.

6. The extraction method according to claim 1, wherein the ultrasonic extraction is conducted with a power of 200 W and a frequency of 40 KHz.

7. The extraction method according to claim 6, wherein the ultrasonic extraction is conducted at 60° C. for 15 minutes to 60 minutes.

8. The extraction method according to claim 1, further comprising:
drying the Hibiscus sabdariffa L. ‘TAITUNG NO. 6’ extract.

9. A Hibiscus sabdariffa L. ‘TAITUNG NO. 6’ extract, characterized in that the Hibiscus sabdariffa L. ‘TAITUNG NO. 6’ extract is prepared by the extraction method as claimed in claim 1.

10. A food composition comprising the Hibiscus sabdariffa L. ‘TAITUNG NO. 6’ extract as claimed in claim 9.

11. A pharmaceutical composition comprising the Hibiscus sabdariffa L. ‘TAITUNG NO. 6’ extract as claimed in claim 9.

12. A skin care cosmetic product comprising the Hibiscus sabdariffa L. ‘TAITUNG NO. 6’ extract as claimed in claim 9.

13. A use of a Hibiscus sabdariffa L. ‘TAITUNG NO. 6’ extract for preparation of a pharmaceutical composition for skin care, characterized in that the Hibiscus sabdariffa L. ‘TAITUNG NO. 6’ extract is prepared by the extraction method as claimed in claim 1.
